(19) United States
(12) Patent Application Publication
(10) Pub. No.: US 2021/0379208 A1
(43) Pub. Date: Dec. 9, 2021

(54) MOLECULAR SENSORS WITH MODIFIED LIGANDS

(71) Applicant: Massachusetts Institute of Technology, Cambridge, MA (US)

(72) Inventors: Alan Pradip Jasanoff, Cambridge, MA (US); He Wei, Arlington, MA (US)

(73) Assignee: Massachusetts Institute of Technology, Cambridge, MA (US)

(21) Appl. No.: 17/224,492

(22) Filed: Apr. 7, 2021

Related U.S. Application Data
(60) Provisional application No. 63/036,002, filed on Jun. 8, 2020.

(51) Int. Cl. A61K 49/18

(52) U.S. Cl. CPC A61K 49/1833

(57) ABSTRACT

The present disclosure generally relates in certain embodiments to a magnetic imaging kit for biochemical sensing, e.g., for detection of analytes, via magnetic imaging techniques. For instance, in one set of embodiments, the kit comprises a collection of magnetic imaging agents (e.g. superparamagnetic iron oxide nanoparticles) and linker species (e.g. a catechol, phosphothreonine, and/or derivative thereof) with a molecular weight of less than or equal to 1000 Da, or less than or equal to 500 Da. The magnetic imaging agents may sense the presence of an analyte via aggregation or disaggregation of the two or more magnetic imaging agents. In addition, certain aspects are directed to a method of exposing two or more magnetic imaging agents to a region on or in a subject and imaging the region to determine the analyte.

### RELATED APPLICATIONS

This application claims priority under 35 U.S.C. § 119(e) to U.S. Provisional Application No. 63/036,002, filed Jun. 8, 2020, and entitled “Molecular Sensors with Modified Ligands,” which is incorporated herein by reference in its entirety for all purposes.

### GOVERNMENT FUNDING

This invention was made with government support under Grant No. R01 DA038642 awarded by the National Institutes of Health. The government has certain rights in the invention.

### FIELD

The present invention generally relates to a magnetic imaging kit, optionally in the biochemical sensing space.

### BACKGROUND

Recent years have seen substantial advances in technology for mapping neurochemical dynamics in the brain. Functional magnetic resonance imaging (functional MRI) has been used as a brain-imaging approach to detect neurophysiology indirectly by detecting changes associated with blood flow. There has been a lack of effort in the development of sensors and probes capable of directly sensing analytes in the brain and being directly imaged by imaging techniques such as MRI. Currently, such probes often produce relatively weak MRI signals, slow response kinetics, and have often been used in a substantial amount that limits sensitivity of analyte detection in vivo. Therefore, there has a need for sensors with better MRI signal, faster response kinetics, and lower application dose for high-sensitivity detection of analytes in the brain for in vivo applications.

### SUMMARY

In certain aspects, a magnetic imaging kit for biochemical sensing is provided.

In some embodiments, the magnetic imaging kit comprises a collection of magnetic imaging agents, optionally magnetic resonance imaging agents; and a surface layer disposed on the magnetic imaging agents, wherein: the surface layer comprises at least one linker species with a molecular weight of less than or equal to 1000 Da, or less than or equal to 500 Da, optionally a catechol, phosphothreonine, and/or derivative thereof, the linker species comprising an immobilization moiety that immobilizes at the surface of the agent and a binding moiety selected to bind an analyte or to bind a binding partner of an analyte, whereby the presence of the analyte affects the proximity of the magnetic imaging agents to each other, and wherein the surface layer has a thickness of less than or equal to 3 nanometers.

In some aspects, a method of magnetic imaging to determine an analyte at a location on or in a subject is provided.

In certain embodiments, the method of magnetic imaging to determine an analyte at a location on or in a subject comprises exposing two or more magnetic imaging agents, optionally magnetic resonance imaging agents, to a region on or in a subject, wherein a proximity relationship between the two or more magnetic imaging agents changes in the presence of an analyte; and imaging a region containing the two or more magnetic imaging agents to determine the analyte, wherein each of the two or more magnetic imaging agents comprises a surface layer disposed on the magnetic imaging agents, wherein: the surface layer comprises at least one linker species with a molecular weight of less than or equal to 1000 Da, or less than or equal to 500 Da, optionally a catechol, phosphothreonine, and/or derivative thereof, the linker species comprising an immobilization moiety that immobilizes at the surface of the agent and a binding moiety selected to bind the analyte or to bind a binding partner of the analyte, whereby the presence of the analyte affects the proximity of the two or more magnetic imaging agents to each other, and wherein the surface layer has a thickness of less than or equal to 3 nanometers.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

Various aspects and embodiments of the application will be described with reference to the following figures. It should be appreciated that the figures are not necessarily drawn to scale.

FIGS. 1A-1F show one or more magnetic imaging agent, in accordance with one embodiment. FIG. 1A shows a magnetic imaging agent with a linker species. FIG. 1B shows a magnetic imaging agent with a plurality of linker species. FIG. 1C shows a magnetic imaging agent with a linker species and a non-linking species. FIG. 1D shows a magnetic imaging agent with a plurality of linker species and a plurality of non-linking species. FIG. 1E shows a magnetic imaging agent with a linker species and an analyte. FIG. 1F shows magnetic imaging agents with linker species and an analog of an analyte or a binding partner of an analyte.

FIGS. 2A-2B show a magnetic imaging kit, in accordance with another embodiment of the disclosure. FIG. 2A and FIG. 2B show the magnetic imaging kit in the absence of an analyte (FIG. 2A) and in the presence of analytes (FIG. 2B).

FIGS. 3A-3C show a magnetic imaging kit, in accordance with some embodiments of the disclosure. FIG. 3A-3C show a magnetic imaging kit (FIG. 3A), the magnetic imaging kit in the absence of an analyte (FIG. 3B), and the magnetic imaging kit in the presence of an analyte (FIG. 3C).

FIG. 4A shows a transmission electron microscopy (TEM) image of superparamagnetic iron oxide nanoparticles, in accordance with some embodiments of the disclosure described herein.

FIGS. 4B-4C show dynamic light scattering measurements of various magnetic imaging kit, in accordance with some embodiments of the disclosure. FIG. 4B shows dynamic light scattering data for MaCaReNa 2.0 magnetic imaging kit. FIG. 4C shows dynamic light scattering data for MS-SPIONs magnetic imaging kit.

FIGS. 5A-5C show synthesis (FIG. 5A) and characterizations (FIG. 5B-5C) of N-maleimido-1-oxo-propyldopamine (MOPD), in accordance with certain embodiments. FIG. 5B-5C show mass spectroscopy characterization (FIG. 5B) and nuclear magnetic resonance characterization (FIG. 5C) of MOPD.

FIGS. 6A-6E show a method of evaluating the sensitivity of a magnetic imaging kit in the presence of an analyte, in accordance with certain embodiments. FIG. 6A shows a schematic of reversible aggregation and disaggregation of a magnetic imaging kit (MaCaReNa 2.0) in the presence of calcium ions. FIG. 6B-6C show dynamic light scattering measurements (FIG. 6B) and magnetic resonance imaging (MRI) measurements (FIG. 6C) of the kit. FIG. 6D shows MRI images at different calcium ion concentration. FIG. 6E shows bio-layer interferometry data monitoring aggregation level of imaging agents in the presence and absence of an anlayte, e.g., calcium ions.

FIGS. 7A-7C show MRI measurements made using a magnetic imaging kit (MaCaReNa 2.0) in a rat's brain, in accordance with certain embodiments. FIG. 7A shows an MRI image of a rat's left thalamus via intraparenchymal injection of the kit. FIG. 7B-7C show MRI images of the rat's brain before (FIG. 7B) and after (FIG. 7C) brain-wide delivery of the kit.

### DETAILED DESCRIPTION

The present disclosure generally relates to magnetic imaging. One aspect relates to a magnetic imaging kit which can be used for biochemical sensing, e.g., for detection of analytes, via magnetic imaging techniques.

In one set of embodiments, a magnetic imaging kit includes magnetic imaging agents with surface layers including a linker species for binding another species, where the linker species is selected to be small and/or to provide a relatively small surface layer thickness, reducing the size of the overall agent and facilitating agent mobility and transport.

For instance, in one set of embodiments, the kit comprises a collection of magnetic imaging agents and at least one linker species with a molecular weight of less than or equal to 1000 Da, or less than or equal to 500 Da. In some embodiments, all linker species associated with a particular magnetic imaging agent have a collective average molecular weight less than those values noted above. The magnetic imaging agents may sense the presence of an analyte via aggregation or disaggregation of the two or more magnetic imaging agents. In addition, certain aspects are directed to a method of exposing two or more magnetic imaging agents to a region on or in a subject and imaging the region to determine the analyte.

In some instances, the biochemical sensing of an analyte may result in a change in a proximity relationship between the magnetic imaging agents. In certain embodiments, by exposing the collection of two or more magnetic imaging agents, e.g., optionally magnetic resonance imaging agents, to a region on or in a subject, a proximity relationship between the two or more magnetic imaging agents changes in the presence of an analyte. The change in the proximity relationship of the magnetic imaging agents may result in a reversible aggregation and/or disaggregation of the magnetic imaging agents in the presence of an analyte. The aggregation and disaggregation kinetics of the collection of magnetic imaging agents may ultimately result in a change in magnetic imaging signals that can be measured by magnetic imaging techniques. Non-limiting examples of magnetic imaging techniques comprise magnetic resonance imaging (MRI) and magnetic particles imaging (MPI). According to certain embodiments, by imaging a region on or in a subject containing the collection of two or more magnetic imaging agents, the analyte can be determined.

For example, in some embodiment, a magnetic imaging kit consists of magnetic imaging agents (e.g., superparamagnetic iron oxide nanoparticles), custom synthesized linker species (e.g., organic ligand coatings), and engineered binder species (e.g., proteins). In the presence of an analyte (e.g., targeted ion or neurotransmitter), the magnetic imaging agents agglomerate to form a larger structure in some cases, or disaggregate from an agglomerated state in other instances. The average hydrodynamic diameter of these magnetic imaging agents may either increase or decrease more than 10 fold, which in turn changes the magnetic coupling state of the magnetic imaging agents (e.g., superparamagnetic iron oxide nanoparticles), leading to several fold changes in the magnetic imaging signal, e.g., MRI or MPI signal.

In some embodiments, the magnetic imaging kit for biochemical sensing comprises a collection of magnetic imaging agents and a surface layer disposed on the magnetic imaging agent that comprises at least one linker species with a molecular weight of less than or equal to 1000 Da, or less than or equal to 500 Da. The linker species comprises an immobilization moiety that immobilizes at the surface of the magnetic imaging agent and a binding moiety selected to bind an analyte or to bind a binding partner of an analyte. For example, depending on the analyte of interest, a specific type of linker species may be selected and disposed in the surface layer of the magnetic imaging agent to either directly or indirectly bind the magnetic imaging agent to the analyte. The magnetic imaging kit may comprise other components, e.g., binder species and/or analogs of the analyte, that may interact with the analyte and participate in the biochemical sensing of the analyte.

In some embodiments, the use of linker species of low molecular weight, e.g., less than or equal to 1000 Da, or less than or equal to 500 Da, results in the formation of an ultra-thin surface layer (e.g., <3 nm) disposed on the magnetic imaging agent. The ultra-thin surface layer may in turn lead to a reduction in the overall size of magnetic imaging agents, thus giving rise to ultra-small magnetic imaging agents. The use of ultra-small magnetic imaging agents in the magnetic imaging kit may advantageously allow for rapid (e.g., single-second or sub-second) and reversible aggregation and disaggregation kinetics in the presence of an analyte. Furthermore, the small size of these magnetic imaging agents allows for ease of delivery to target regions that may otherwise be difficult to access. For example, as a non-limiting example, these ultra-small magnetic imaging agents may give rise to better pharmacokinetics, e.g., distribution of magnetic imaging agents, in the animal brain by using either locally intraparenchymal injection or brain-wide cerebrospinal fluid (CSF) injection method, which allows for mapping analyte (e.g., ions and neurotransmitters) concentration and studying neural circuits in the whole animal brain.

In some embodiments, the magnetic imaging kit for biochemical sensing described herein comprises a collection of two or more magnetic imaging agents. In some such embodiments, the magnetic imaging agents comprise iron oxide nanoparticles. A non-limiting example of a magnetic imaging agent is a superparamagnetic iron oxide nanoparticle, e.g., SPION. It should be understood that the magnetic imaging agents are not limited to SPIONs, and that any magnetic imaging agents capable of being detected via magnetic imaging techniques (e.g., MRI or MPI) may be used. Other non-limiting examples include MRI and/or MPI contrast agents comprising paramagnetic gadolinium, superparamagnetic iron platinum, superparamagnetic manganese, protein-based MRI contrast agents, etc.

According to some embodiment, the magnetic imaging kit comprises a surface layer disposed on the magnetic imaging agents. In some embodiments, the surface layer comprises at least one linker species with a relatively low molecular weight of less than or equal to 4000 Da, less than or equal to 3000 Da, less than or equal to 2000 Da, less than or equal to 1000 Da, less than or equal to 900 Da, less than or equal to 800 Da, less than or equal to 700 Da, less than or equal to 600 Da, or less than or equal to 500 Da, less than or equal to 400 Da, less than or equal to 350 Da, less than or equal to 325 Da, less than or equal to 300 Da, less than or equal to 250 Da, less than or equal to 200 Da, less than or equal to 150 Da, or less than or equal to 100 Da. In some embodiments, the surface layer comprises at least one linker species with a molecular weight of greater than or equal to 50 Da, greater than or equal to 100 Da, greater than or equal to 150 Da, greater than or equal to 200 Da, greater than or equal to 250 Da, greater than or equal to 300 Da, greater than or equal to 325 Da, greater than or equal to 350 Da, greater than or equal to 400 Da, greater than or equal to 500 Da, greater than or equal to 600 Da, greater than or equal to 700 Da, greater than or equal to 800 Da, greater than or equal to 900 Da, greater than or equal to 1000 Da, greater than or equal to 2000 Da, or greater than or equal to 3000 Da. Combination of the above-referenced ranges are possible (e.g., greater than or equal to 100 Da and less than or equal to 1000 Da, or greater than or equal to 100 Da and less than or equal to 500 Da.) Other ranges are also possible.

In some embodiments, all linker species in the surface layer (associated with a particular magnetic imaging agent) have a collective average molecular weight in one or more of the above-referenced ranges.

In some embodiments, the linker species is a small molecule. The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, not more than 300 g/mol, not more than 200 g/mol, or not more than 100 g/mol. In certain embodiments, the molecular weight of a small molecule is at least 100 g/mol, at least 200 g/mol, at least 300 g/mol, at least 400 g/mol, at least 500 g/mol, at least 600 g/mol, at least 700 g/mol, at least 800 g/mol, or at least 900 g/mol, or at least 1,000 g/mol. Combinations of the above ranges (e.g., at least 200 g/mol and not more than 500 g/mol) are also possible.

In some embodiments, the linker species is an oligomer or a polymer that has a relatively low average molecular weight in one or more of the ranges described above. The term “average molecular weight” may encompass the number average molecular weight (Mn), weight average molecular weight (Mw), higher average molecular weight (Mz or Mz+1), GPC/SEC-determined average molecular weight (Mp), and viscosity average molecular weight (Mv). In certain embodiments, the average molecular weight is Mw. In certain embodiments, the Mn is determined with gel permeation chromatography, viscometry via the (Mark-Houwink equation), colligative methods (such as vapor pressure osmometry), end-group determination, or proton NMR. In certain embodiments, the Mw is determined with static light scattering, small angle neutron scattering, X-ray scattering, and sedimentation velocity.

Non-limiting examples of linker species with a molecular weight described herein comprises a catechol, a phosphothreonine, a carboxylic acid, an amine, a dextran, and/or derivative thereof. For example, a linker species comprising a catechol derivative may comprise N-maleimido-1-oxo-propyldopamine (MOPD). It should be understood that the linker species are not limited to those described herein, as long as the linker species has a molecular weight and capabilities described herein.

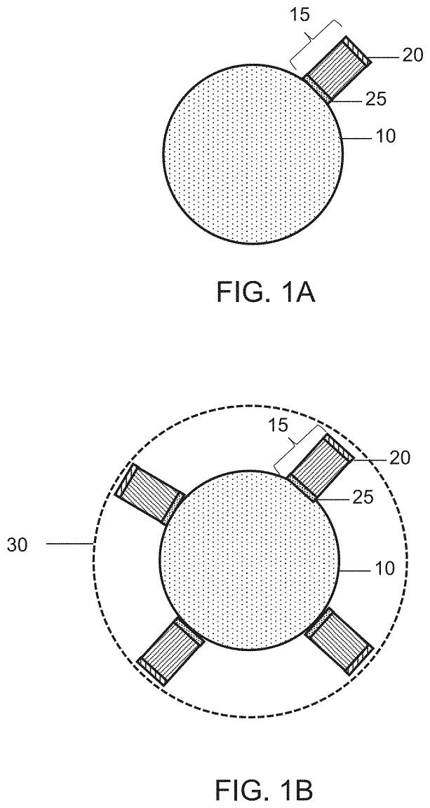

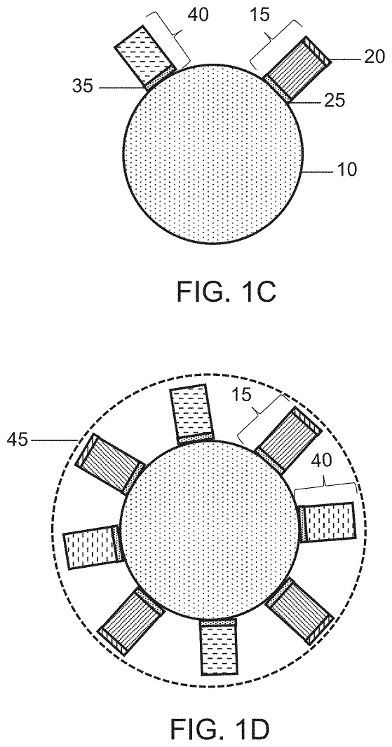

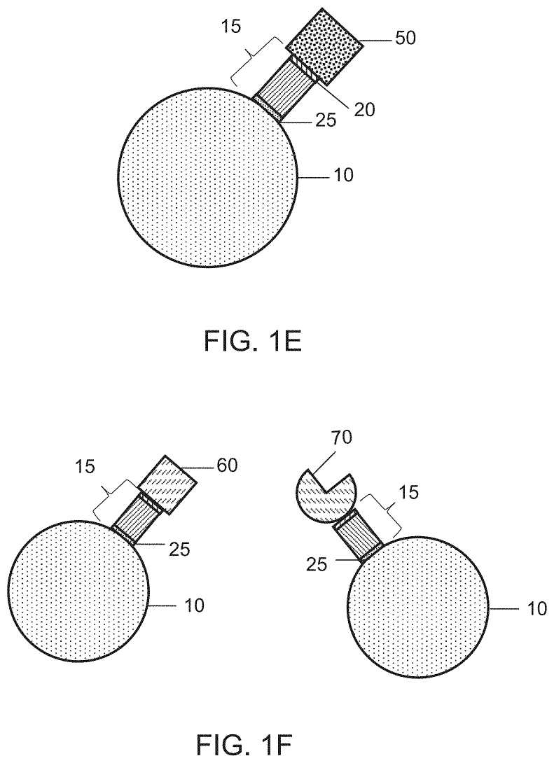

In addition, in accordance with certain embodiments, the linker species comprises an immobilization moiety that immobilizes at the surface of the magnetic imaging agent (e.g., a SPION) and a binding moiety selected to bind an analyte or to bind a binding partner of an analyte. A non-limiting representation of a linker species 15 is illustrated in FIG. 1A, where the linker species comprises an immobilization moiety 25 that immobilizes the linker species 15 at the surface of the magnetic imaging agent 10 and a binding moiety 20 positioned at a side of the linker species 15 opposite the side of the linker species containing the immobilization moiety 25. In accordance with certain embodiments, FIG. 1B illustrates an imaging agent 10 comprising a surface layer 30 having at least one linker species 15 immobilized at the surface of the imaging agent 10. As noted above, the figures are schematic and are not intended to be drawn to scale. It is understood that linker species, binding moieties, and the like will not appear, in reality, the way they appear as will illustrated schematically in connection with this disclosure.

In accordance to certain embodiments, the immobilization moiety comprises molecular constructs or a species that facilitate binding or immobilization of a linker species to the surface of the magnetic imaging agents by either physical and/or chemical means. For instance, for magnetic imaging agents such as superparamagnetic iron oxide nanoparticles (SPIONs), non-limiting examples of immobilization moiety that can be used include catechol, amines, carboxylic acids, phosphates, etc., as long as the immobilization moiety is capable of providing coordination to the surface of the magnetic imaging agents (e.g., iron oxide surface of SPIONs). In one set of embodiments, a specific type of catechols, e.g., dopamine, has been used as the immobilization moiety in a custom synthesized small molecule linker species, e.g., N-maleimido-1-oxo-propyldopamine (MOPD), to immobilize the linker species at the surface of the superparamagnetic iron oxide nanoparticles (SPIONs). In another set of embodiments, a small molecular weight linker species, e.g., phosphothreonine, comprises a threonine group that assists with immobilization of the linker species at the surface of the superparamagnetic iron oxide nanoparticles (SPIONs).

In some embodiments, the linker species comprises a binding moiety selected to bind an analyte, a binding partner of an analyte, or optionally an analog of an analyte, by either physical or chemical means. The specific chemistry or type of binding moiety in the linker species depends on the specific chemistry of the target species, e.g., the specific analyte, the specific binding partner of an analyte, or specific analog of the analyte. According to certain embodiments, the linker species comprises a binding moiety that may bind to a target species by physical means (e.g., adsorption, absorption, etc.). For instance, in accordance with certain embodiments, for an ionic analyte (e.g., a calcium ion), a linker species (e.g., phosphothreonine) comprising a binding moiety (e.g., the phosphate group) may be selected to coordinate to the ionic analyte (e.g., a calcium ion, etc.) via coordination chemistry. According to certain embodiments, the linker species may comprise a binding moiety that binds to a target species via chemical means (e.g., a chemical reaction). For instance, in accordance with certain embodiments, a custom synthesized linker species (e.g., N-maleimido-1-oxo-propyldopamine (MOPD)) comprising a binding moiety (e.g., a maleimide group) may be selected to bind a target species containing a thiol group via thiol-maleimide conjugation. It should be noted that any appropriate chemistries and reactions (e.g., click reactions) may be used to bind the binding moiety to an analyte, a binding partner of an analyte, or an analog of an analyte.

Additional examples of binding moieties include, but are not limited to, hydroxyl, thiol, amino, carboxylic acid, maleimide, phosphate, alkyne, azide, or combination thereof.

According to certain embodiments, the surface layer comprises at least one linker species and at least one non-linking species. For instance, the non-linking may be immobilized at the surface of a magnetic imaging agent to stabilize or disperse the magnetic imaging agent in solution. The non-linking species comprises an immobilization moiety that immobilizes the linking species at the surface of the magnetic imaging agent. FIG. 1C illustrates a non-limiting representation a magnetic imaging agent 10 comprising a linker species 15 and a non-linking species 40. The non-linking species 40 comprises an immobilization moiety 35 that immobilizes the non-linking species 40 at the surface of a magnetic imaging agent 10. The linker species 15 illustrated in FIG. 1C may have similar properties to the linker species from FIG. 1A-1B. According to certain embodiments, the immobilization moiety on the non-linking species may either be the same as to or different from the immobilization moiety on the linker species. For example, a non-limiting example of the immobilization moiety on the non-linking species may comprise a catechol, e.g., a dopamine, that is capable of coordinating to the surface of a magnetic imaging agent, e.g., superparamagnetic iron oxide nanoparticle (SPION). In accordance with certain embodiment, for example, the non-linking species comprises N-sulfopropyl-N,N-dimethyldopamine (SDD) or derivatives thereof.

Additional examples of non-linking species include, but are not limited to N-sulfopropyl-N,N-dimethyldopamine derivatives comprising any of a variety of immobilization moieties such as phosphates, carboxylic acids, polyhydroxy groups, and/or amines.

In some embodiments, the non-linking species may have a relatively low molecular weight. For example, the non-linking species may have a molecular weight (or average molecular weight) in one or more of the ranges of molecular weight described above with respect to the linker species. For example, the non-linking species may have a molecular weight of between 100 Da and 1000 Da, or between 100 Da and 500 Da.

In some instances, the magnetic imaging agents comprises a surface layer with a thickness of less than or equal to 3 nm. In some embodiments, the surface layer has a thickness of less than or equal to 10 nm, less than or equal to 8 nm, less than or equal to 6 nm, less than or equal to 4 nm, less than or equal to 2 nm, etc. In addition, the surface layer, in some cases, may have a thickness of at least 1 nm, at least 3 nm, at least 5 nm, at least 7 nm, at least 9 nm, etc. Combinations of these are also possible; for example, the surface layer may have a thickness of between 1 nm and 4 nm. The surface layer disposed on the magnetic may comprise at least one linker species and/or at least one non-linking species. Non-limiting representations of a surface layer are illustrated in FIG. 1B and FIG. 1C. FIG. 1B illustrate a surface layer 30 comprising linker species 15; FIG. 1C illustrates a surface layer 45 comprising both linker species 15 and non-linking species 40.

The thickness of the surface layer may be measured using any of a variety of appropriate techniques including, but are not limited to, SEM, TEM, x-ray scattering, neutron scattering, Atomic Force Microscopy, and/or Size Exclusion Chromatography.

In some instances, the magnetic imaging agent comprises an overall size (e.g., hydrodynamic diameter) that is small enough to allow for rapid (e.g., single-second or sub-second) proximity change (e.g., aggregation and/or disaggregation kinetics) of the magnetic imaging agents in the presence of an analyte. The overall size (e.g., hydrodynamic diameter) of a magnetic imaging agent may be dictated by the size of the magnetic imaging agent (e.g., a superparamagnetic iron oxide nanoparticles (SPION)) as well as the additional thickness attributed by the surface layer disposed on the magnetic imaging agent.

In some instances, taking into account the surface layer thickness, the overall size (e.g., hydrodynamic diameter) of a magnetic imaging agent may be less than or equal to 10 nanometers. In some embodiments, the overall size of a magnetic imaging agent may be less than or equal to 50 nm, less than or equal to 45 nm, less than or equal to 40 nm, less than or equal to 35 nm, less than or equal to 30 nm, less than or equal to 25 nm, less than or equal to 20 nm, less than or equal to 15 nm, less than or equal to 10 nm, less than or equal to 5 nm, etc. In addition, the overall size of a magnetic imaging agent, in some cases, may be at least 3 nm, at least 8 nm, at least 13 nm, at least 18 nm, at least 23 nm, at least 28 nm, at least 33 nm, at least 38 nm, at least 43 nm, at least 48 nm, etc. Combinations of these are also possible; for example, a magnetic imaging agent may have an overall size of between 3 nm and 10 nm.

In accordance with some embodiments, the magnetic imaging kit further comprises at least one binder species, e.g., additional components that assist with sensing and detecting of an analyte. In some embodiments, the kit comprises at least one binder species having multiple binding sites for an analyte, e.g., an ionic analyte. The specific type of binder species is dictated by the analyte of interest. Some non-limiting examples of binder species comprise proteins, peptides, aptamers, custom-synthesized organic molecules, etc.

A “protein,” “peptide,” or “polypeptide” comprises a polymer of amino acid residues linked together by peptide bonds. The term refers to proteins, polypeptides, and peptides of any size, structure, or function. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. In certain embodiments, the amino acid residues of a peptide are alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine, in D and/or L form. In certain embodiments, the amino acid residues of a peptide are alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine, in L form. One or more of the amino acids in a protein may be protected. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. In certain embodiments, a protein comprises between 2 and 10, between 10 and 30, between 30 and 100, between 100 and 300, or between 300 and 1,000, inclusive, amino acids. In certain embodiments, a protein comprises between 1,000 and 3,000, or between 3,000 and 10,000, inclusive, amino acids. In certain embodiments, the amino acids in a protein are natural amino acids. In certain embodiments, the amino acids in a protein are unnatural amino acids. In certain embodiments, the amino acids in a protein are a combination of natural amino acids and unnatural amino acids.

The disclosure is not intended to be limited in any manner by the above exemplary listing of substituents. Additional terms may be defined in other sections of this disclosure.

According to certain embodiments, provided herein is a method of magnetic imaging to determine an analyte at a location on or in a subject by using the magnetic imaging kit described elsewhere herein. In some instances, the analyte comprises an ionic analyte. Non-limiting examples of the ionic analyte may be an ion (e.g., a calcium ion, a potassium ion, etc.), a charged molecule, a charged polymer, etc. According to certain embodiments, the analyte comprises a molecular ligand, e.g., a neurotransmitter. Non-limiting examples of a neurotransmitter comprise dopamine, serotonin, norepinephrine, GABA, acetylcholine (ACh), glutamate, etc. In some instances, the location on or in a subject may be an organ within the subject such as the brain. For example, the magnetic imaging kit may be used to sense an analyte (e.g., calcium ion, dopamine, serotonin, etc.) within the brain. It should be understood that the use of the kit for biochemical sensing is not limited to use at a location on or in a subject (e.g., in vivo), the kit could be used outside of a subject or in any suitable environment (e.g., ex vivo) that contains an analyte of interest. A non-limiting illustration of an analyte is presented in FIG. 1E. As shown, a magnetic imaging agent 10 can bind to an analyte 50 via the binding moiety 20 of linking species 15.

In accordance with certain embodiments, by exposing the collection of two or more magnetic imaging agents, e.g., optionally magnetic resonance imaging agents, to a region on or in a subject, a proximity relationship between the two or more magnetic imaging agents changes in the presence of an analyte. The proximity relationship between the two or more magnetic imaging agents may either increase via aggregation of the magnetic imaging agents or decrease via disaggregation of the magnetic imaging agents.

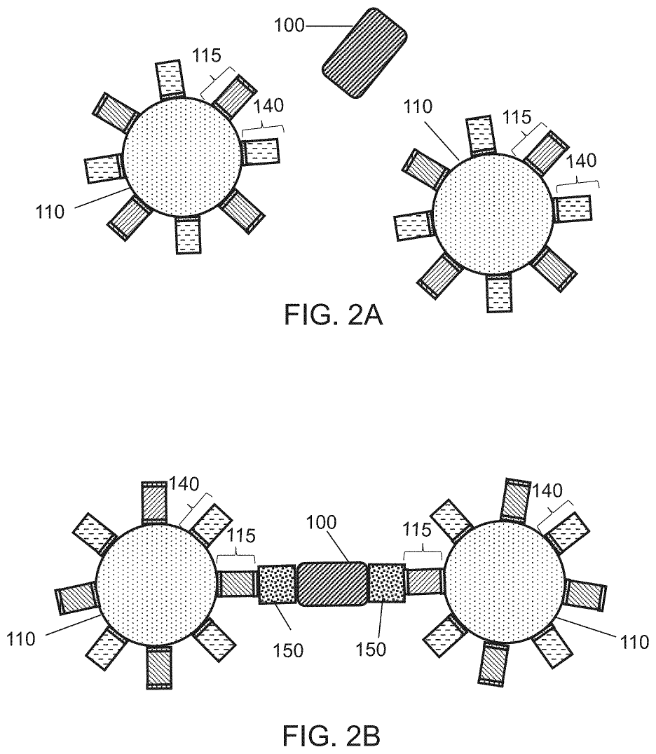

In some instances, according to certain embodiments, the magnetic imaging kit comprises at least one binder species having multiple binding sites for the analyte (e.g., ionic analyte), such that the collection of magnetic imaging agents aggregate via the binder species in the presence of the analyte (e.g., ionic analyte). In some such embodiments, the magnetic imaging kit comprises a collection of two or more magnetic imaging agents, at least one linker species, at least one binder species, and optionally at least one non-linking species. A non-limiting representation of the kit is shown in FIG. 2A, which shows a binder species 100 and a collection of identical magnetic imaging agents 110 comprising identical linker species 115 and non-linking species 140. In some embodiment, the proximity relationship between the two or more magnetic imaging agents increases via aggregation of the magnetic imaging agents in the presence of the analyte. A non-limiting representation of this aggregation is shown in FIG. 2B. As shown, the collection of two magnetic imaging agent 110 from FIG. 2A aggregate via the binder species 100 in the presence of the analyte 150. According to certain embodiments, aggregation of the magnetic imaging agents occurs as a result of binding of the binder species and linkers to the analyte, e.g., thus giving rise to the formation of a linker-analyte-binder-analyte-linker complexation between the magnetic imaging agents. FIG. 2B illustrates such a linker 115-analyte 150-binder 100-analyte 150-linker 115 complexation formed between the collection of two identical imaging agents 110. Although FIG. 2A-2B illustrates a collection of two identical magnetic imaging agents with identical linker species and non-linking species, it should be noted that the collection may comprise two or more different magnetic imaging agents, as well as different linker species and non-linking species, depending on the application needs.

A non-limiting example of a kit for sensing an ionic analyte, e.g., calcium ions, is described herein. The magnetic imaging kit comprises a collection of two or more magnetic imaging agents (e.g., superparamagnetic iron oxide nanoparticles (SPIONs)), at least one linker species (e.g., phosphothreonine, and/or derivatives thereof), at least one binder species (e.g., a calcium-binding protein such as C2AB), and at least non-linking species (e.g., N-sulfopropyl-N,N-dimethyldopamine (SDD)). The calcium binding protein C2AB may comprise two calcium-binding domains. In the presence of the ionic analyte, e.g., calcium ions, the two or more superparamagnetic iron oxide nanoparticles (SPIONs) aggregate via a reversible linker-analyte-binder-analyte-linker complexation formed between the superparamagnetic iron oxide nanoparticles (SPIONs). Again, in this example, FIG. 2B may be used to illustrate the linker 115-analyte 150-binder 100-analyte 150-linker 115 complexation formed between the collection of two identical imaging agents (e.g., SPIONs) 110.

In some embodiments, the proximity relationship between the two or more magnetic imaging agents decreases via disaggregation of the magnetic imaging agents. According to certain embodiments, the magnetic imaging kit comprises a collection of two or more magnetic imaging agents, where the collection of two or more magnetic imaging agents comprises magnetic imaging agents from a first population of magnetic imaging agents and from a second population of magnetic imaging agents. In some such embodiments, the two populations of imaging agents are configured to bind different target species, e.g., an analog of the analyte or a binding partner of the analyte, via the linker species. For instances, each magnetic imaging agent from the first population of imaging agent is bounded to at least one binding partner of the analyte via the linker species, and each magnetic imaging agent from the second population of magnetic imaging agents is bounded to at least one analog of the analyte via the linker species. A non-limiting representation of the embodiment is now shown in FIG. 1F, where the collection of magnetic imaging agents comprises a collection of two identical imaging agents 10 and identical linker species 15. As shown, while one magnetic imaging agent is bounded to an analog of the analyte 60 via linker species 15, the other imaging agent is bounded to a binding partner of the analyte 70 via linker species 15. Although FIG. 1F illustrates a collection of two identical magnetic imaging agents with identical linker species, it should be understood that the two magnetic imaging agents may be different and that linker species on one imaging agent may also differ from the linker species on another imaging agent.

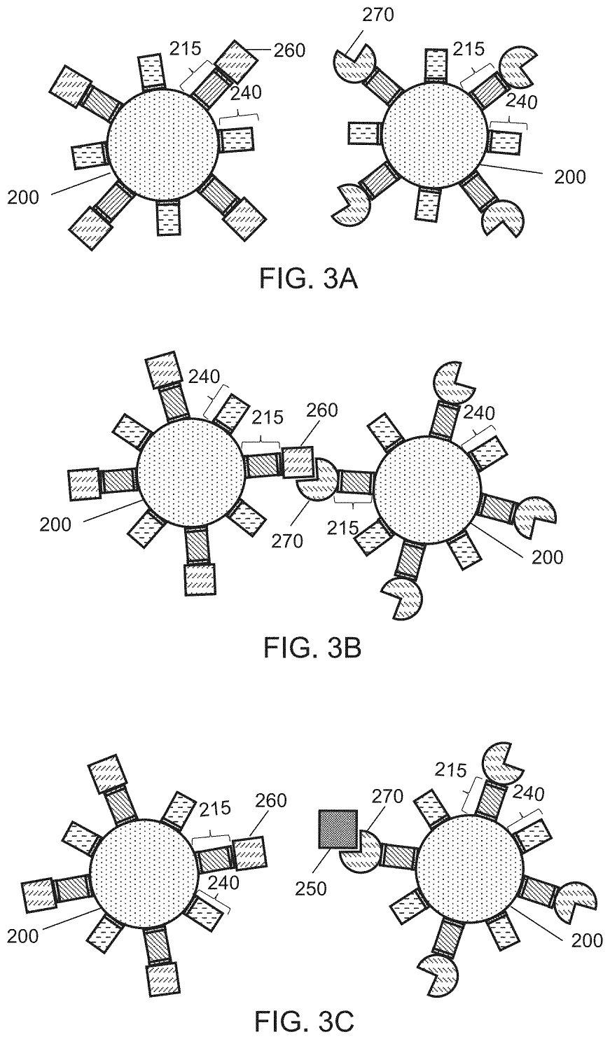

According to some embodiments, in the absence of the analyte, the second population of magnetic imaging agents with surface-bound analogs of the analyte are aggregated with the first population of magnetic agents with surface-bound binding partners of the analyte. A non-limiting representation of the two population of magnetic imaging agent is shown in FIG. 3A, where the collection of magnetic imaging agents comprises two identical magnetic imaging agent 200 with identical linker species 215. While one magnetic imaging agent comprises surface-bound analogs of the analyte 260, the other magnetic imaging agent comprises surface-bound binding partners of the analyte 270. Non-linking species 240 may be optionally present on the surface of both imaging agents 200. In some embodiments, in the absence of the analyte, the first and second population of magnetic imaging agents bind to each other via association of the surface-bounds analogs of the analyte with the surface-bound binding partners of the analyte. As shown in FIG. 3B, the identical magnetic imaging agents 200 from FIG. 3A are aggregated via an association of a surface-bound analog of the analyte 260 on one magnetic imaging agent with a surface-bound binding partner of the analyte 270 on the other magnetic imaging agent. Although FIG. 3A-3C illustrates a collection of two identical magnetic imaging agents with identical linker species, it should be understood that the two magnetic imaging agents may be different and that linker species on one imaging agent may different from the linker species on another imaging agent.

According to certain embodiments, in the presence of the analyte, the second population of magnetic imaging agents with surface-bound analogs of the analyte are disaggregated from the first population of magnetic agents with surface-bound binding partners of the analyte, as the analyte competitively binds to the surface-bound binding partners of the analyte on the first population of magnetic imaging agents. FIG. 3C is a non-limiting representation of the embodiment. In the presence of an analyte 250, the magnetic imaging agent with surface-bound analogs of the analyte 260 is disaggregated from the magnetic imaging agent with surface-bound binding partners of the analyte 270 as the analyte 250 competitively binds to a surface-bound binding partner of the analyte 270. In some instances, the binding affinity of the analyte with the binding partner of the analyte may be much larger than the binding affinity of the analog of the analyte with the binding partner of the analyte.

A non-limiting example of a magnetic imaging kit for sensing a neurotransmitter, e.g., dopamine or serotonin, is described herein. The magnetic imaging kit comprises a collection of two or more magnetic imaging agents (e.g., superparamagnetic iron oxide nanoparticles (SPIONs)), at least one linker species (e.g., a custom synthesized catechol comprising N-maleimido-1-oxo-propyldopamine (MOPD)), and at least one non-linking species (e.g., N-sulfopropyl-N,N-dimethyldopamine (SDD)). The collection of two or more magnetic imaging agents (e.g., superparamagnetic iron oxide nanoparticles (SPIONs)) comprises at least one magnetic imaging agent from a first population of magnetic imaging agents (SPIONs) with surface-bound binding partners of analyte, e.g., proteins capable of binding with the neurotransmitter, and at least one magnetic imaging agent from a second population of magnetic imaging agents (SPIONs)) with surface-bound analogs of the analyte, e.g., neurotransmitter analogs. The binding partners of the analyte and/or the analogs of the analyte may be chemically modified (e.g., to contain a thiol-modified group) to allow for conjugation to a moiety (e.g., maleimide moiety) on the linker species (e.g., N-maleimido-1-oxo-propyldopamine (MOPD)). Any appropriate chemistries and reactions (e.g., click chemistry) may be used.

According to certain embodiments, before exposing the collection of two or more imaging agents (SPIONs) to an analyte (e.g., neurotransmitter), the two or more magnetic imaging agents in the kit are aggregated via binding of the surface-bound analog of analyte (e.g., neurotransmitter analog) to the surface-bound binding partner of an analyte (e.g., binding protein of the neurotransmitter). As the magnetic imaging kit is exposed to the presence of the analyte (e.g., neurotransmitter), the analyte (e.g., neurotransmitter) competitively binds to the binding partner of the analyte (e.g., binding protein of the neurotransmitter) on the first population of magnetic imaging agent, which disrupts the binding between the analogs of the analyte and the binding partners of the analyte, thus resulting in the disaggregation of the collection of two of more magnetic imaging agents.

Non-limiting examples of a binding partner of an analyte include proteins, peptides, polypeptides, calcium-binding proteins and peptides, dopamine-binding proteins and peptides, serotonin binding proteins and peptide, etc.

Certain embodiments comprise imaging a region containing the two or more magnetic imaging agents to determine the analyte. By using the magnetic imaging kit described herein, the presence of an analyte may be sensed and the type and/or amount of the analyte may be determined via magnetic imaging techniques. For instance, as described herein, the proximity relationship between the two or more magnetic imaging agents changes in the presence of an analyte. The change in proximity may manifest itself in a change in average size, e.g., average hydrodynamic diameter, of a collection of imaging agents, as a result of aggregation or disaggregation of the magnetic imaging agents. This measure of size is at a lower value when in the form of a single imaging agent (or average size of dispersed imaging agents), and a larger value when a cluster of imaging agents is formed. For instance, in the presence of the analyte, the change in average size of the resultant clusters, as compared with average size of dispersed magnetic imaging agents, may be more than 5 fold, more than 10 fold, or more than 20 fold. The change in proximity relationship between the two or more magnetic imaging agents may be detected by a change in magnetic imaging signals, and the amount and/or type of the analyte may be determined from the change in magnetic imaging signals.

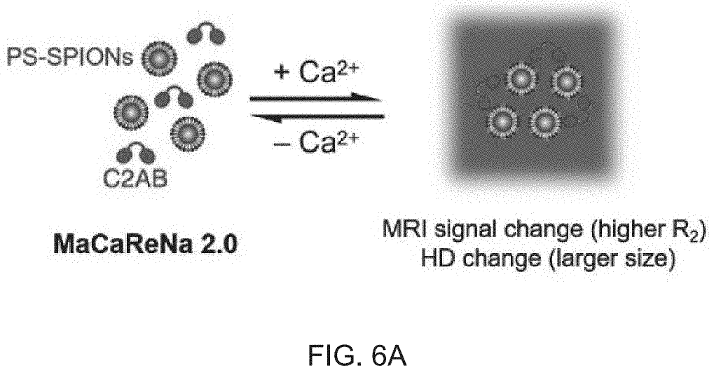

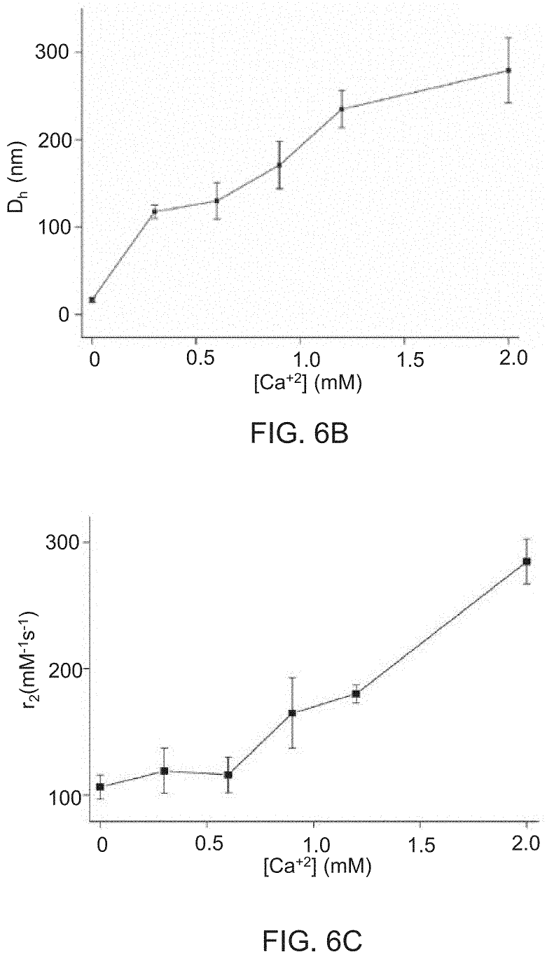

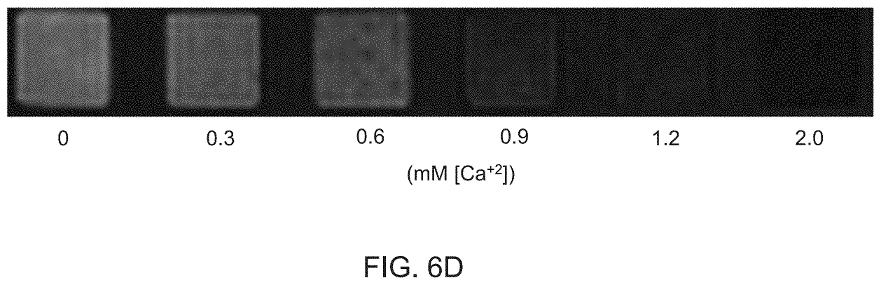

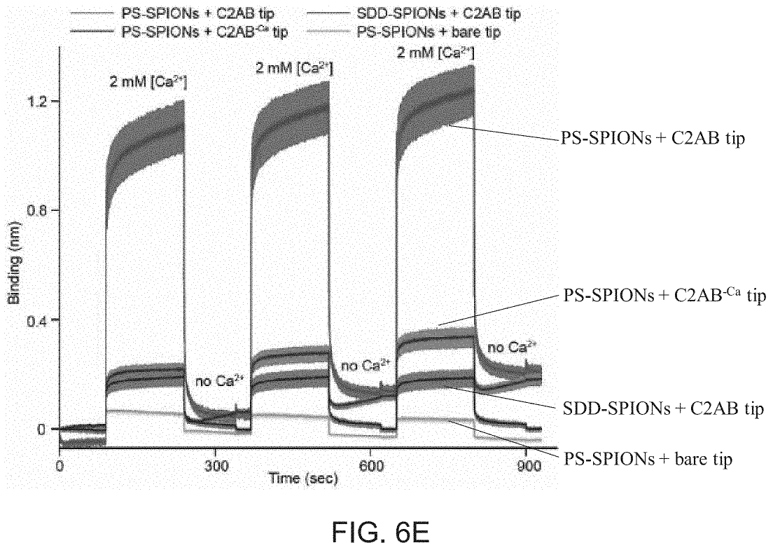

A non-limiting example of determining an ionic analyte, e.g., calcium ions, using magnetic resonance imaging (MRI) is described herein. As described elsewhere herein and illustrated in FIG. 2B, in the presence of the ionic analyte, e.g., calcium ions, the two or more superparamagnetic iron oxide nanoparticles (SPIONs) aggregate via a linker-analyte-binder-analyte-linker complexation formed between the superparamagnetic iron oxide nanoparticles (SPIONs). The proximity relationship between the two or more superparamagnetic iron oxide nanoparticles (SPIONs) increases as the nanoparticles aggregate to form a larger structure. The size change associated with the superparamagnetic iron oxide nanoparticles (SPIONs) due to aggregation results in a change in the MRI signal (e.g., r2 relaxivity). In accordance with certain embodiments, the magnitude of size change and/or change in MRI signal may be used to determine an amount of the analyte present. For example, as shown in FIG. 6C, the specific amount of an ionic analyte, e.g., calcium ions, may be correlated to a MRI signal, e.g., r2 relaxivity. In accordance with some embodiments, the specific amount of an ionic analyte, e.g., calcium ions, can be determined by the change in MRI signal and/or the change in average size (e.g., average hydrodynamic diameter) of the magnetic imaging agents.

According to some embodiments, the proximity change between the imaging agents is reversible. For instance, the magnetic imaging agent kit undergoes reversible aggregation and disaggregation kinetics in the presence or absence of analyte. For example, as illustrated by the example in FIG. 2B in accordance to one embodiment, in the presence of an analyte, the magnetic imaging agents form a larger structure via the formation of a linker-analyte-binder-analyte-linker complexation. In some embodiments, with the removal of the analyte, the complexation dissociates and the magnetic imaging agents disaggregates.

In some embodiments, a change in the proximity relationship between the two or more magnetic imaging agents in the presence of an analyte occurs at a single second or sub-second scale. The change in the proximity relationship comprises aggregation and/or disaggregation of the two or more magnetic imaging agents in the presence of an analyte. In some instances, the change in the proximity relationship between the two or more magnetic imaging agents in the presence of an analyte occurs at less than or equal to 5 seconds, or optionally less than or equal to 1 second. In some embodiments, the change in the proximity relationship between the two or more magnetic imaging agents in the presence of an analyte occurs at less than or equal to 50 seconds, less than or equal to 40 seconds, less than or equal to 30 seconds, less than or equal to 20 seconds, less than or equal to 10 seconds, less than or equal to 5 seconds, less than or equal to 4 seconds, less than or equal to 3 seconds, less than or equal to 2 seconds, less than or equal to 1 second, less than or equal to 0.8 seconds, less than or equal to 0.6 seconds, less than or equal to 0.4 seconds, less than or equal to 0.2 seconds, less than or equal to 0.1 seconds, etc.

In some such embodiments, a magnetic imaging agent kit comprising a surface layer and linkers species with molecular weight of less than or equal to 1000 Da, or less than or equal to 500 Da, may be used to bring about a rapid (e.g., single-second, or sub-second) change in the proximity relationship of the magnetic imaging agents. In some embodiments, a magnetic imaging agent kit comprising a surface layer less than or equal to 3 nm may be used to bring about a rapid (e.g., single-second, or sub-second) change in the proximity relationship of the magnetic imaging agents in the presence of an analyte. In some embodiments, a magnetic imaging agent kit comprising a surface layer less than or equal to 3 nm and linkers species with molecular weight of less than or equal to 1000 Da, or less than or equal to 500 Da, may be used to bring about a rapid (e.g., single-second, or sub-second) change in the proximity relationship of the magnetic imaging agents in the presence of an analyte.

Certain embodiments comprise a method for intraparenchymal injection of the magnetic imaging agent kit described elsewhere herein to a subject to sense an analyte, followed by magnetic imaging of the region containing the imaging agent kit to determine the analyte. In some embodiments, the magnetic imaging agent kit is delivered through the cerebrospinal fluid (CSF) for brain-wide sensing of an analyte, followed by determination of the analyte using magnetic imaging techniques. It should be understood that the region of injection is not limited to the brain or limited to in vivo applications only; the magnetic imaging kit may be applied to other regions on or in a subject as well as for application outside of the subject.

The following examples are intended to illustrate certain embodiments of the present disclosure, but do not exemplify the full scope of the disclosure.

### Example 1

The synthesis, characterization, and application of ion and neurotransmitter sensitive magnetic imaging kits are described in the following examples. These kits consisted of magnetic imaging agents (e.g., superparamagnetic iron oxide nanoparticle), custom synthesized linker species and/or non-linking species (e.g., organic ligand coatings), and binder species (e.g., engineered proteins). In the presence of targeted ion or neurotransmitter, the average hydrodynamic diameter of the collection of surface-coated imaging agents increased more than 10 fold, which in turn changed the magnetic coupling state of the magnetic agents, leading to several fold changes in magnetic resonance imaging (MRI) signal and resulting in expected changes in magnetic particle imaging (MPI) signal. Moreover, the collection of surface-coated imaging agents demonstrated subsecond and reversible aggregation/disaggregation kinetics in the presence or absence of targeted ion. Furthermore, the kits could spread reasonably well in the animal brain by using locally intraparenchymal injection or brain-wide cerebrospinal fluid (CSF) injection method, thus paving the way for mapping ion/neurotransmitter concentration and studying neural circuits in the whole animal brain.

This example illustrates a method for synthesizing magnetic imaging kits that are used to sense and determine various analytes, in accordance with certain embodiments of the invention. Each imaging agent (e.g., superparamagnetic iron oxide nanoparticle) comprises a surface layer comprising linker species (e.g., phosphothreonine (PT) or N-maleimido-1-oxo-propyldopamine (MOPD)), in accordance with certain embodiments of the invention. Optionally, the surface layer of each imaging agent also includes non-linker species (e.g., N-sulfopropyl-N,N-dimethyldopamine (SDD)). The kit optionally further comprise binder species (e.g., calcium-sensitive protein C2AB).

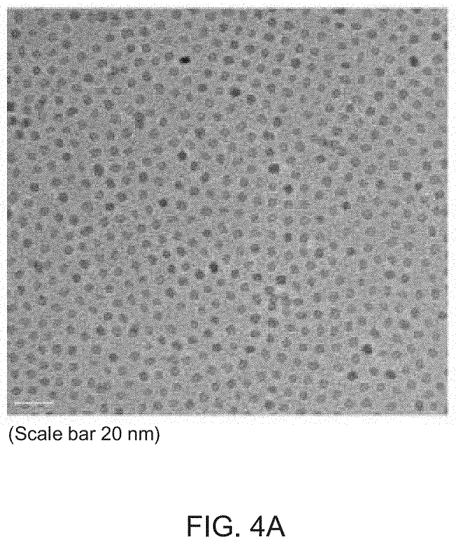

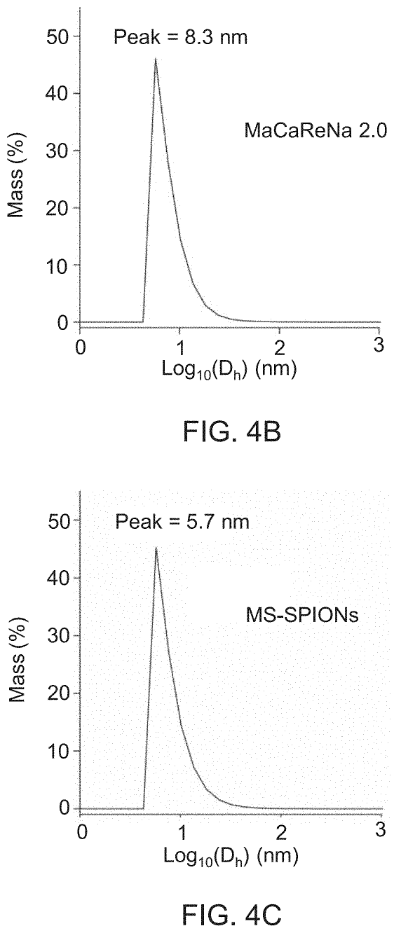

A method for the synthesis of calcium-sensitive, dopamine-sensitive, and serotonin-sensitive magnetic imaging kits, which consist of superparamagnetic iron oxide nanoparticles (FIG. 4A), N-sulfopropyl-N,N-dimethyldopamine (SDD), and a secondary ligand coating including phosphothreonine (PT) or N-maleimido-1-oxo-propyldopamine (MOPD) is presented herein.

Phosphothreonine (PT) and N-sulfopropyl-N,N-dimethyldopamine (SDD) coated superparamagnetic iron oxide nanoparticles (SPIONs) were prepared for calcium sensing as described herein. First, 20 mg PT was dissolved in 0.5 mL of 2 mol/L, 4-(2-hydroxyethyl)-1-piperazineethanesulfonic acid (HEPES) buffer that has a pH of 7.5, followed by the addition of 1 mL of SDD-coated SPIONs (SDD-SPIONs, 50 mM [Fe]). The reaction mixture was allowed to stir at room temperature (RT) for 18 hours and was then dialyzed thoroughly by using 10 kDa Amicon™ centrifugal units, in order to remove the unreacted PT. This yielded PT and SDD coated SPIONs (PS-SPIONs), which were then mixed with calcium-sensitive protein C2AB, leading to the formation of magnetic calcium-responsive nanoparticles version 2.0 (MaCaReNa 2.0). Dynamic light scattering (DLS) measurement results showed that MaCaReNa 2.0 had a mass-based average hydrodynamic diameter (HD) of 8.3 nm (FIG. 4B).

MOPD and SDD coated SPIONs for dopamine and serotonin sensing were prepared as described herein. First, 3.83 mg MOPD was dissolved in 0.05 mL of 2 mol/L, 4-(2-hydroxyethyl)-1-piperazineethanesulfonic acid (HEPES) buffer that has a pH of 7.5, followed by the addition of 1 mL of SDD-coated SPIONs (SDD-SPIONs, 50 mM [Fe]). The reaction mixture was allowed to stir at 4° C. for 18 hours and was then dialyzed thoroughly by using 10 kDa Amicon™ centrifugal units, in order to remove the unreacted MOPD. This yielded MOPD and SDD coated SPIONs (MS-SPIONs). DLS measurement results showed that MS-SPIONs had a mass-based average hydrodynamic diameter of 5.7 nm (FIG. 4C).

### Example 2

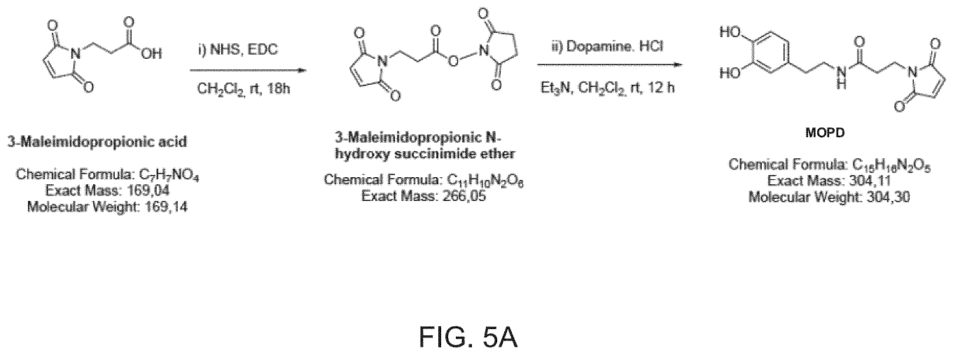

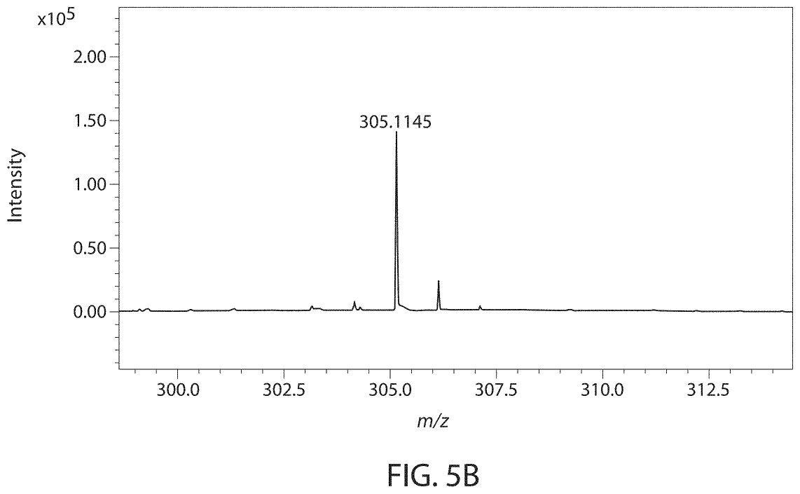

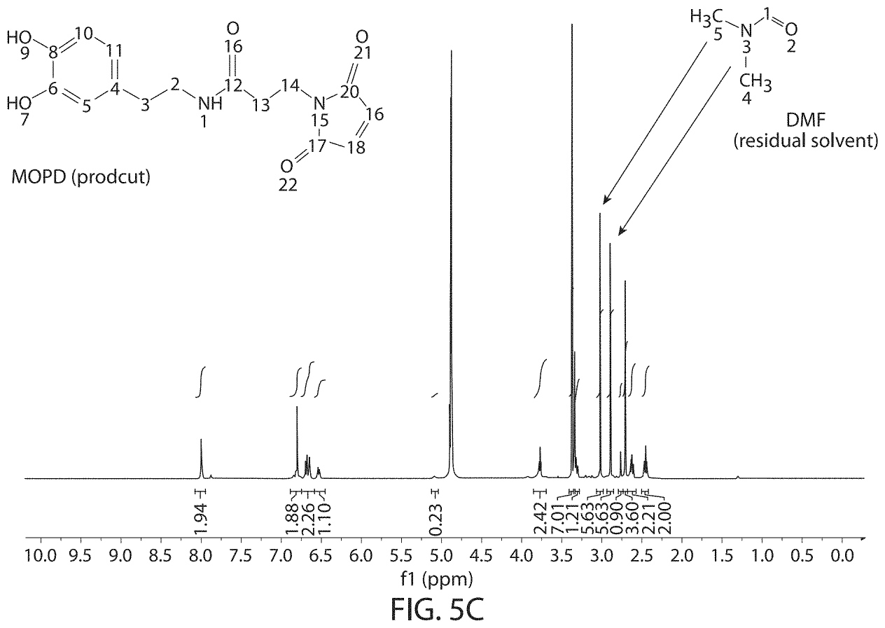

This example illustrates a method for synthesizing a specific linker species, e.g., the linker species used in the MS-SPIONs as described in Example 1. Specifically, a method for the synthesis (FIG. 5A) and characterization (FIG. 5B-5C) of N-maleimido-1-oxo-propyldopamine (MOPD) is presented herein.

Synthesis of MOPD was performed as shown in FIG. 5A. First, 1 g of 3-maleimido propionic acid, 1.13 g of 1-Ethyl-3-(3-dimethylaminopropyl)carbodiimide (EDC), and 0.679 g of N-Hydroxysuccinimide (NHS) were dissolved in 100 mL of anhydrous dichloromethane. The reaction mixture was stirred under Argon gas at room temperature for 18 hours. Afterwards, 1.1 g of dopamine hydrochloride and 0.8 mL of triethylamine were added in succession into the above reaction mixture, along with 10 mL of anhydrous dichloromethane (DCM) and 12 mL of anhydrous dimethylformamide (DMF). The reaction mixture was again allowed to stir under Argon gas at room temperature for 12 hrs. Upon the completion of the reaction, the mixture was concentrated by using rotary evaporator to remove the solvents, yielding the crude product, which was then purified by silica gel column chromatography. The eluting solvent used was a mixture of hexanes and acetone (from 1:1 to 1:2).

Mass spectroscopy characterization of as-synthesized MOPD is shown in FIG. 5B. As suggested by the m/z peak at 305.1, the presence of MOPD had been successfully detected by mass spectroscopy. Nuclear magnetic resonance (NMR) characterization of MOPD was also performed to detect the presence of MOPD, as shown in FIG. 5C.

### Example 3

This example illustrates the change in proximity relationship of the magnetic imaging agents to each other in the presence of the analyte, in accordance with some embodiments of the invention. Specifically, the analyte of interest in this example is calcium ions and the MaCaReNa 2.0 magnetic imaging kit described in Example 1 was used to sense and determine the calcium ions.

A method for evaluating the sensitivity of as-synthesized kit (e.g., MaCaReNa 2.0 from Example 1) based on the responses of their average hydrodynamic diameter (HD), magnetic resonance imaging (MRI) signal, and magnetic particle imaging (MPI) signal to calcium ions (FIG. 6A) is presented herein. As shown in FIG. 6A, the collection of PT and SDD coated SPIONs (i.e., PS-SPIONs) in the kit would aggregate in the presence of calcium ions via the C2AB calcium binding proteins, and disaggregate in the absence of calcium ions. A combination of dynamic light scattering (DLS) and In vitro MRI measurements was performed to monitor the aggregation kinetics of the PS-SPIONs magnetic imaging agents in the kit in the presence of calcium ions.

Dynamic light scattering (DLS) measurements was used to measure the average hydrodynamic diameter of the MaCaReNa 2.0 when exposed to different concentrations of calcium ions. The average hydrodynamic diameter (HD) given by DLS is a direct measurement of the aggregation level of the collection of PT and SDD coated SPIONs (PS-SPIONs) in the presence of calcium ions with different physiological concentrations in the range of 0-2 mM (FIG. 6B). As shown in FIG. 6B, the average hydrodynamic diameter of the MaCaReNa 2.0 increased with an increase in calcium ion concentration, indicating aggregation of the collection of PS-SPIONs in the presence of calcium ions. In vitro MRI was performed to measure the T₂ contrast power, i.e. r₂ relaxivity, of MaCaReNa 2.0 in different calcium concentrations (FIG. 6C). The r₂ relaxivity of these MaCaReNa 2.0 was shown to increase with the increment of calcium concentration, leading to darkened MRI images at high calcium concentration (FIG. 6D).

While DLS was used to reflect the aggregation level of magnetic imaging agents (e.g., PS-SPIONs) in equilibrium state, bio-layer interferometry (BLI) was used to characterize the kinetics and reversibility of the aggregation/disaggregation of magnetic imaging agents (e.g., PS-SPIONs) in the presence and absence of calcium ions, separately (FIG. 6E). The tip of BLI biosensor was coated with different binder species: i) C2AB calcium binding protein, ii) C2AB-Ca, a variant of C2AB protein that is incapable of calcium binding, or iii) a bare tip without any coating. The tip of BLI biosensor with a specific binder species was immersed in a solution containing SPIONs with specific coatings. The SPIONs may be PS-SPIONs (SPIONs coated with phosphothreonine (PT) and N-sulfopropyl-N,N-dimethyldopamine (SDD)), or SDD-SPIONs (SPIONs coated with N-sulfopropyl-N,N-dimethyldopamine (SDD)). The binding and unbinding kinetics of four SPIONs and tip combination in the presence/absence of calcium ion was monitored, as shown in FIG. 6E. In the presence of calcium ions, the PS-SPIONs+C2AB tip demonstrated drastically higher binding values compared to PS-SPIONs+C2AB^{-Ca} tip, SDD-SPIONs+C2AB tip, and PS-SPIONs+bare tip (the control). The BLI result suggested that the phosphothreonine linker species on PS-SPIONs and the calcium binding protein C2AB could interact with calcium ions on a subsecond time scale. Additionally, the PS-SPIONs+C2AB was demonstrated to show reversible aggregation/disaggregation kinetics. At different time points, as calcium ions were either introduced to or removed from the system, the PS-SPIONs+C2AB tip exhibited reversible binding and unbinding kinetics.

Magnetic particle imaging (MPI): MaCaReNa 2.0, MS-SPIONs, and their derivatives with similar compositions all contained superparamagnetic iron oxide nanoparticles (SPIONs) and thus can be detected by the MPI technique. Moreover, the aggregation/disaggregation status of these magnetic imaging agents (SPIONs) in the kits (MaCaReNa 2.0, MS-SPIONs) is expected to change the MPI signal accordingly.

### Example 4

This example illustrates methods of exposing a collection (e.g., two or more) of molecular imaging agents to a region on or in a subject, in accordance with certain embodiments of the invention. Specifically, the MaCaReNa 2.0 described in Example 1 was injected into a rat's brain to determine an analyte, e.g., calcium ions.

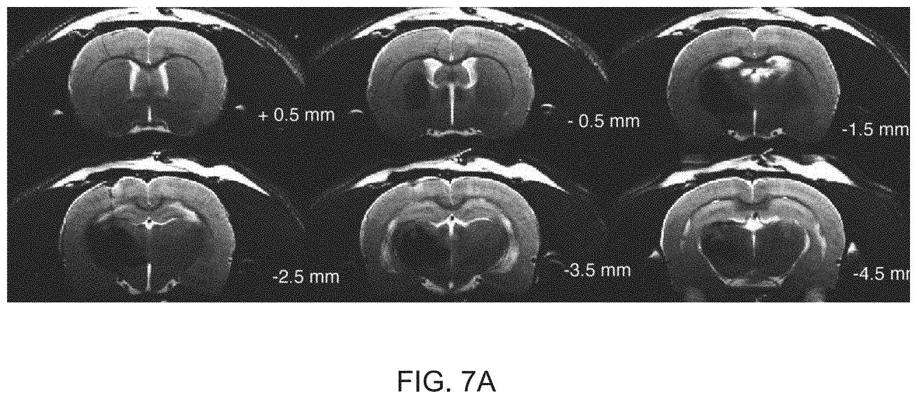

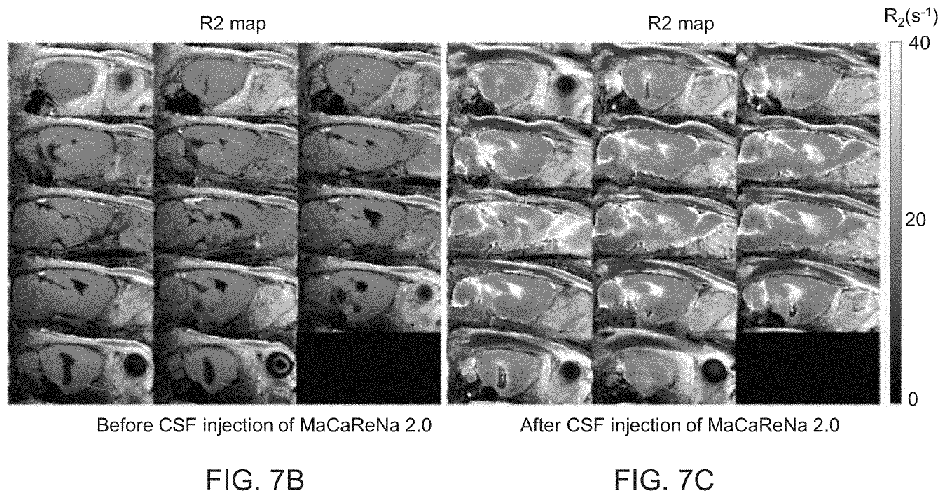

A method for the intraparenchymal injection as well as brain-wide delivery (through the cerebrospinal fluid route) of as-synthesized kit from Example 1 is presented herein. MaCaReNa 2.0 were injected into a rat's left thalamus via intraparenchymal injection, followed by T₂-weighted MRI measurements (FIG. 7A). As the MaCaReNa 2.0 kit was injected into the rat's left thalamus, the kit could be used to sense and determine the presence and concentration of calcium ions via aggregation of the PS-SPIONs, as described in detail in Example 3. The tissue permeability of MaCaReNa 2.0 was reflected in the contrast of the image in FIG. 7A, e.g., where darkened regions suggested the spreading of MaCaReNa 2.0.

MaCaReNa 2.0 kit was delivered through the cerebrospinal fluid (CSF) route for brain-wide delivery. The R₂ maps of all sagittal slices throughout the rat brain are shown for both before CSF injection and after CSF injection of the kit (FIG. 7A-7B). Several regions of different contrast in the R₂ maps were observed in the after CSF injection R₂ maps (FIG. 7C) compared to the before CFS injection R₂ maps (FIG. 7B), which suggested presence of calcium ions in those regions with different contrasts. Based on the contrast in those regions, the concentration of calcium ions was determined.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A magnetic imaging kit for biochemical sensing, comprising:
a collection of magnetic imaging agents; and
a surface layer disposed on the magnetic imaging agents, wherein:
the surface layer comprises at least one linker species with a molecular weight of less than or equal to 1000 Da, the linker species comprising an immobilization moiety that immobilizes at the surface of the agent and a binding moiety selected to bind an analyte or to bind a binding partner of an analyte, whereby the presence of the analyte affects the proximity of the magnetic imaging agents to each other, and
wherein the surface layer has a thickness of less than or equal to 3 nanometers.

2. A method of magnetic imaging to determine an analyte at a location on or in a subject, comprising:
exposing two or more magnetic imaging agents, to a region on or in a subject, wherein a proximity relationship between the two or more magnetic imaging agents changes in the presence of an analyte; and
imaging a region containing the two or more magnetic imaging agents to determine the analyte,
wherein each of the two or more magnetic imaging agents comprises a surface layer disposed on the magnetic imaging agents, wherein:
the surface layer comprises at least one linker species with a molecular weight of less than or equal to 1000 Da, the linker species comprising an immobilization moiety that immobilizes at the surface of the agent and a binding moiety selected to bind the analyte or to bind a binding partner of the analyte, whereby the presence of the analyte affects the proximity of the two or more magnetic imaging agents to each other, and
wherein the surface layer has a thickness of less than or equal to 3 nanometers.

3. The magnetic imaging kit of claim 1, wherein the magnetic imaging agents are magnetic resonance imaging agents.

4. The magnetic imaging kit of claim 1, wherein the at least one linker species has a molecular weight of less than or equal to 500 Da.

5. The magnetic imaging kit of claim 1, wherein the linker species comprises one or more of a catechol, a phosphothreonine, and/or derivative thereof.

6. The magnetic imaging kit of claim 1, wherein the analyte comprises an ionic analyte.

7. The magnetic imaging kit of claim 6, wherein the ionic analyte comprises a calcium ion.

8. The magnetic imaging kit of claim 1, wherein the kit further comprises at least one binder species having multiple binding sites for the analyte, and wherein the collection of magnetic imaging agents aggregate via the binder species in the presence of the analyte.

9. The magnetic imaging kit of claim 8, wherein the linker species comprises phosphothreonine and/or derivative thereof.

10. The magnetic imaging kit of claim 1, wherein the analyte comprises a neurotransmitter.

11. The magnetic imaging kit of claim 1, where the collection of magnetic imaging agents comprises a first population of magnetic imaging agents and a second population of magnetic imaging agents.

12. The magnetic imaging kit of claim 11, wherein each magnetic imaging agent from the first population of magnetic imaging agents is bounded to at least one binding partner of the analyte via the linker species.

13. The magnetic imaging kit of claim 12, wherein each magnetic imaging agent from the second population of magnetic imaging agents is bounded to at least one analog of the analyte via the linker species.

14. The magnetic imaging kit of claim 11, wherein the linker species comprises a catechol derivative comprising N-maleimido-1-oxo-propyldopamine (MOPD).

15. The magnetic imaging kit of claim 13, wherein in the absence of the analyte, the second population of magnetic imaging agents with surface-bound analog of the analyte are aggregated with the first population of magnetic agents with surface-bound binding partner of the analyte.

16. The magnetic imaging kit of claim 1, wherein the surface layer further comprises a non-linking species comprising N-sulfopropyl-N,N-dimethyldopamine (SDD).

17. The magnetic imaging kit of claim 1, wherein the magnetic imaging agents comprise superparamagnetic iron oxide nanoparticles.

18. The magnetic imaging kit of claim 1, wherein a change in the proximity relationship between the two or more magnetic imaging agents in the presence of an analyte occurs in less than or equal to 5 seconds, or optionally less than or equal to 1 second.

19. The method of magnetic imaging of claim 2, wherein the linker species comprises one or more of a catechol, a phosphothreonine, and/or derivative thereof.

20. The method of magnetic imaging of claim 2, wherein the at least one linker species has a molecular weight of less than or equal to 500 Da.

21. The method of magnetic imaging of claim 2, wherein the magnetic imaging agents are magnetic resonance imaging agents.
